(19) United States
(12) Patent Application Publication
(10) Pub. No.: US 2026/0325223 A1
(43) Pub. Date: Oct. 22, 2026

(54) ENDOSCOPY DEVICE AND ASSEMBLY METHOD THEREOF

(71) Applicants: Remex Medical Corp., TAICHUNG CITY (TW); Remex Medical USA Inc., Diamond Bar, CA (US)

(72) Inventors: Iou-Sheng CHANG, Taichung City (TW); Guan-Ting LIN, Taichung City (TW); Kuan-Hsun WANG, Taichung City (TW)

(21) Appl. No.: 19/650,305

(22) Filed: Apr. 16, 2026

Related U.S. Application Data
(60) Provisional application No. 63/790,752, filed on Apr. 18, 2025.

(30) Foreign Application Priority Data
Mar. 18, 2026 (TW) 115110282

(51) Int. Cl. A61B 34/20; A61B 1/00

(52) U.S. Cl. CPC A61B 34/20; A61B 1/0011; A61B 1/00135; A61B 2034/2055

(57) ABSTRACT

An endoscopy device is configured corresponding to an optical tracker and includes an endoscope, a sleeve assembly and a dynamic reference assembly. The endoscope has a first end and a second end, and the first end is opposite to the second end. The sleeve assembly is detachably disposed at the first end of the endoscope. The sleeve assembly includes a sleeve, and the sleeve is curved. The dynamic reference assembly is rotatably positioned at the second end of the endoscope and configured to be detected by the optical tracker to obtain a position of the endoscope. When the sleeve assembly is disposed at the first end of the endoscope, the second end of the endoscope is away from the sleeve assembly, and the sleeve assembly sleeves the first end of the endoscope.

### RELATED APPLICATIONS

This application claims priority to U.S. Provisional Application Serial Number 63/790,752, filed Apr. 18, 2025; and Taiwan Application Serial Number 115110282, filed Mar. 18, 2026, which are herein incorporated by reference.

### BACKGROUND

### Field of Invention

The present disclosure relates to an endoscopy device and an assembly method thereof, and more particularly to an endoscopy device and an assembly method thereof that facilitate a physician in finding orientation and direction during use.

### Description of Related Art

Endoscopy devices are widely applied in industrial and medical fields, enabling a user to view narrow locations that cannot be observed by the naked eye, particularly having a significant impact on the medical field. A physician can extend an endoscope into a human body to observe internal conditions via the endoscopy device, thereby facilitating further diagnosis and judgment.

Conventional endoscopy devices utilize a straight-tube endoscope and a cannula. Such a structure makes it difficult for the physician to identify the correct orientation and direction during an actual operation, thereby reducing operational convenience. The viewing angle of the endoscope (commonly referred to as Direction of View (DOV)) is a critical parameter in clinical procedures. Common viewing angles include 0 degrees, 30 degrees, 45 degrees and 70 degrees. The provision of an angled lens is primarily based on the following considerations: (1) Overcoming anatomical “blind spots”. The interior of the human body is not a straight and unobstructed passage, but rather includes numerous curves and obstructions, such as the spine, joint cavities, and nasal passages. “0-degree lens (direct view)” permits visualization only in a forward direction, similar to a flashlight. In contrast, “angled lens (oblique view)” enables visualization of regions located around corners or behind structures without requiring substantial tilting of the entire endoscope, such that anatomical regions which are otherwise not directly visible may be observed. For example, in spinal surgery, the angled lens allows the physician to observe a lateral aspect of a nerve root without requiring excessive lowering of the endoscope shaft to avoid bony structures. (2) Achieving panoramic sweep by rotation. When the 0-degree lens is used, rotation of the endoscope shaft does not alter the image content. In contrast, when the angled lens is used, rotation of the endoscope shaft causes the direction of view to sweep around a central axis so as to define a conical viewing range. Moreover, the physician may rotate the endoscope shaft in situ at a single incision site to observe the entire surrounding cavity without requiring large-scale displacement of the endoscope as a stirring motion, so that damage to patient tissue can be minimized. (3) Avoiding instrument clash. In minimally invasive surgery, the incision space is extremely limited, and the endoscope and surgical instruments (such as hemostats and drills) are often positioned in close proximity. When the 0-degree lens is used, the endoscope is required to be aligned directly with the lesion, thereby obstructing the operational path of the surgical instruments. In contrast, when the angled lens is used, the endoscope may be introduced from a lateral direction at an oblique angle while maintaining a line of sight toward a central surgical target, such that the handle and camera head of the endoscope are spatially offset from a hand operating space of the physician, so that instrument collision can be avoided.

However, the diameter of conventional angled lens (particularly chip-on-the-tip angled lens) increases with an increase in the viewing angle. In view of the above, there is currently a lack of an endoscopy device and an assembly method thereof that facilitate a physician in finding orientation and direction during use. Therefore, relevant practitioners are seeking solutions thereto.

### SUMMARY

According to an implementation of a structural aspect of the present invention, provided is an endoscopy device configured corresponding to an optical tracker and including an endoscope, a sleeve assembly and a dynamic reference assembly. The endoscope has a first end and a second end, and the first end is opposite to the second end. The sleeve assembly is detachably disposed at the first end of the endoscope. The sleeve assembly includes a sleeve, and the sleeve is curved. The dynamic reference assembly is rotatably positioned at the second end of the endoscope and configured to be detected by the optical tracker to obtain a position of the endoscope. When the sleeve assembly is disposed at the first end of the endoscope, the second end of the endoscope is away from the sleeve assembly, and the sleeve assembly sleeves the first end of the endoscope.

According to an implementation of a method aspect of the present invention, provided is an assembly method of an endoscopy device, including: installing a sleeve assembly onto a first end of an endoscope; and installing a dynamic reference assembly onto a second end of the endoscope, such that the sleeve assembly, the endoscope, and the dynamic reference assembly are assembled and fixed to each other. The endoscopy device is configured corresponding to an optical tracker, the first end is opposite to the second end, the sleeve assembly is detachably disposed at the first end of the endoscope, the sleeve assembly includes a sleeve, the sleeve is curved, the second end of the endoscope is away from the sleeve assembly, the sleeve assembly sleeves the first end of the endoscope, and the dynamic reference assembly is rotatably positioned at the second end of the endoscope, and is configured to be detected by the optical tracker to obtain a position of the endoscope.

### BRIEF DESCRIPTION OF THE DRAWINGS

The present disclosure can be more fully understood by reading the following detailed description of the embodiment, with reference made to the accompanying drawings as follows:

FIG. 1 is a schematic diagram illustrating an endoscopy device according to a first embodiment of the present disclosure.

FIG. 2 is an exploded view of the endoscopy device of FIG. 1.

FIG. 3 is a partial schematic diagram illustrating a dynamic reference assembly and the endoscope of the endoscopy device of FIG. 1.

FIG. 4 is a cross-sectional view of the endoscopy device of FIG. 1 taken along line 4-4.

FIG. 5 is a cross-sectional view of the endoscopy device of FIG. 1 taken along line 5-5.

FIG. 6A is a cross-sectional view of the endoscopy device of FIG. 1 taken along line 6A-6A.

FIG. 6B is a cross-sectional view of the endoscopy device of FIG. 1 taken along line 6B-6B.

FIG. 7 is a schematic flowchart illustrating an assembly method of an endoscopy device according to a second embodiment of the present invention.

FIG. 8 is a flowchart illustrating one step of the assembly method of the endoscopy device of FIG. 7.

FIG. 9 is a schematic assembly diagram illustrating the step of FIG. 8.

### DETAILED DESCRIPTION

The embodiment will be described with the drawings. For clarity, some practical details will be described below. However, it should be noted that the present disclosure should not be limited by the practical details, that is, in some embodiment, the practical details is unnecessary. In addition, for simplifying the drawings, some conventional structures and elements will be simply illustrated, and repeated elements may be represented by the same labels.

It will be understood that when an element (or device) is referred to as be “connected to” another element, it can be directly connected to the other element, or it can be indirectly connected to the other element, that is, intervening elements may be present. In contrast, when an element is referred to as be “directly connected to” another element, there are no intervening elements present. In addition, the terms first, second, third, etc. are used herein to describe various elements or components, these elements or components should not be limited by these terms. Consequently, a first element or component discussed below could be termed a second element or component.

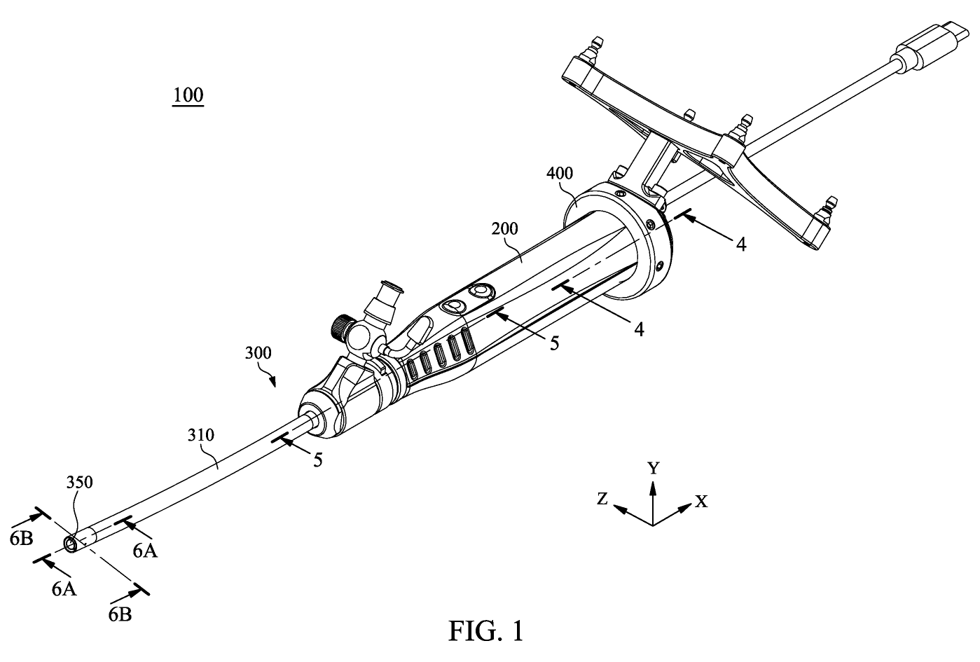

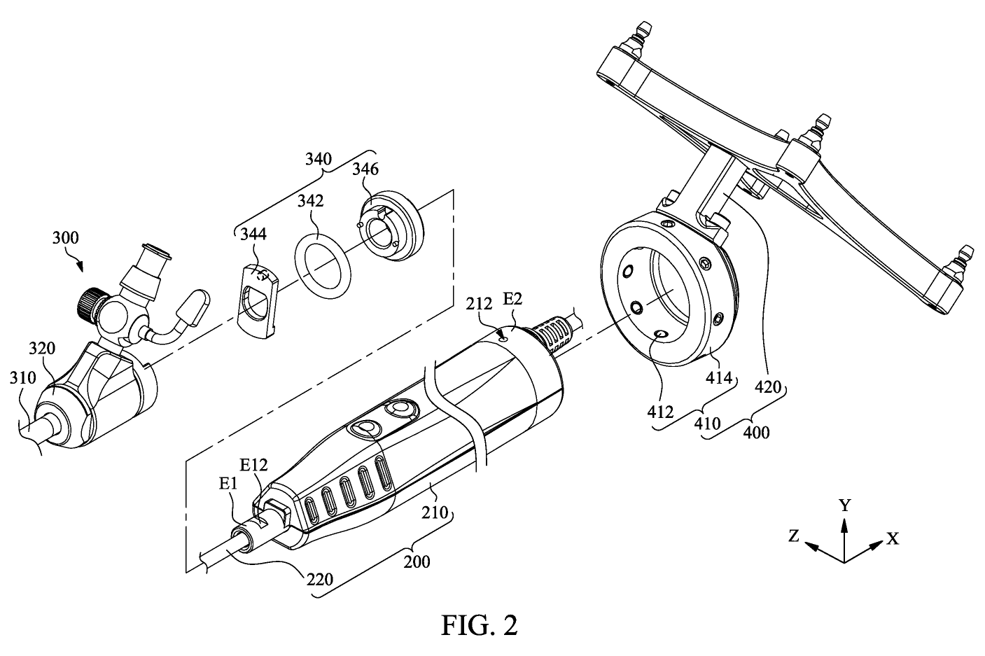

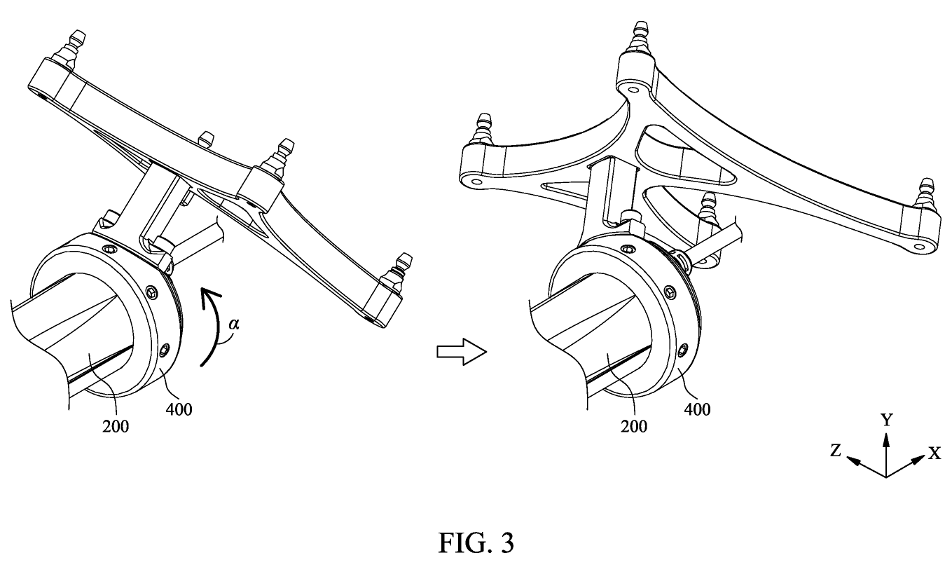

Reference is made to FIG. 1, FIGS. 2 and 3 together, where FIG. 1 is a schematic diagram illustrating an endoscopy device 100 according to a first embodiment of the present disclosure; FIG. 2 is an exploded view of the endoscopy device 100 of FIG. 1; and FIG. 3 is a partial schematic diagram illustrating a dynamic reference assembly 400 and the endoscope 200 of the endoscopy device 100 of FIG. 1. The endoscopy device 100 can be configured corresponding to an optical tracker. The endoscopy device 100 includes an endoscope 200, a sleeve assembly 300 (a sheath), and a dynamic reference assembly 400. The endoscope 200 has a first end E1 (a front end) and a second end E2 (a rear end), and the first end E1 is opposite to the second end E2. The sleeve assembly 300 is detachably disposed at the first end E1 of the endoscope 200, and the sleeve assembly 300 includes a sleeve 310. The sleeve 310 is curved (the sleeve 310 is a curved tube). The dynamic reference assembly 400 is rotatably positioned at the second end E2 of the endoscope 200, and is configured to be detected by the optical tracker to obtain a position of the endoscope 200. When the sleeve assembly 300 is disposed at the first end E1 of the endoscope 200, the second end E2 of the endoscope 200 is away from the sleeve assembly 300, and the sleeve assembly 300 sleeves the first end E1 of the endoscope 200.

Specifically, the endoscope 200 includes a body 210 and an endoscope tube 220. The body 210 has the first end E1 and the second end E2. The endoscope tube 220 is connected to the first end E1 of the body 210 and is curved (the endoscope tube 220 is a curved tube). The shape of the endoscope tube 220 is the same as (identical to) that of the sleeve 310. When the dynamic reference assembly 400 is positioned at the second end E2 of the endoscope 200, the dynamic reference assembly 400 is detected by the optical tracker to obtain the position of the endoscope 200.

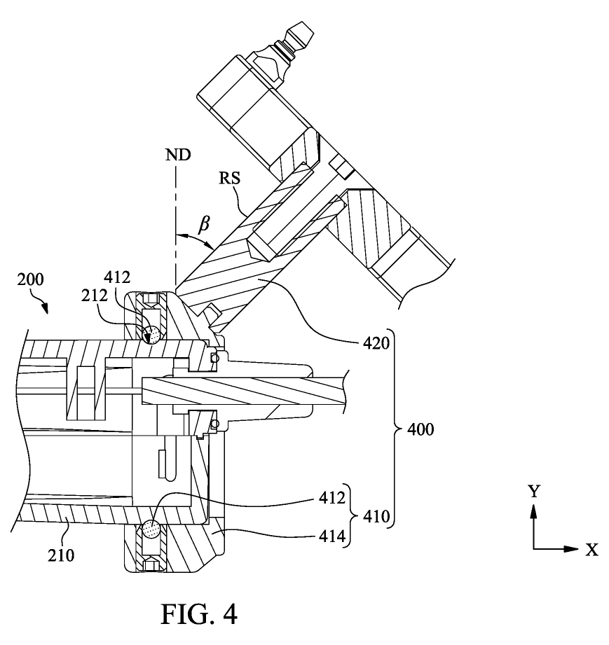

Reference is made to FIGS. 1, 2, 3 and 4 together, where FIG. 4 is a cross-sectional view of the endoscopy device 100 of FIG. 1 taken along line 4-4. The dynamic reference assembly 400 includes a rotating ring 410 and a dynamic reference frame (DRF) 420. The rotating ring 410 is rotatable by an angle α along a YZ plane. The dynamic reference frame 420 is connected to the rotating ring 410 and has a reference surface RS. The endoscope 200 has a normal direction ND (same as Y-axis direction). An included angle β is formed between the reference surface RS and the normal direction ND, and the included angle β is greater than 0 degrees and less than 90 degrees (an acute angle). The rotating ring 410 includes at least one positioning bead 412. The second end E2 of the endoscope 200 includes a groove 212. When the dynamic reference assembly 400 is positioned at the second end E2 of the endoscope 200, the at least one positioning bead 412 is correspondingly embedded in the groove 212. Therefore, the present invention utilizes the rotatable dynamic reference assembly 400, allowing the user to adaptively adjust the direction of the dynamic reference frame 420 according to their own usage habits, thereby significantly increasing the convenience of operation.

The number of the at least one positioning bead 412 is plural, and the rotating ring 410 further includes an annular body 414. The positioning beads 412 are spaced apart in the annular body 414, and the dynamic reference frame 420 is fixedly connected to the annular body 414. In one embodiment, the number of the positioning beads 412 is eight, the angle α can be equal to 45 degrees, and the included angle β can be equal to 45 degrees. In other embodiments, the number of the grooves 212 at the second end E2 of the endoscope 200 can be plural (for example, the number of the grooves 212 can be two, and the two grooves 212 can be respectively disposed on opposite two sides of the second end E2). The more the number of the grooves 212 is, the better the engagement effect between the second end E2 and the rotating ring 410 is. However, the present invention is not limited thereto.

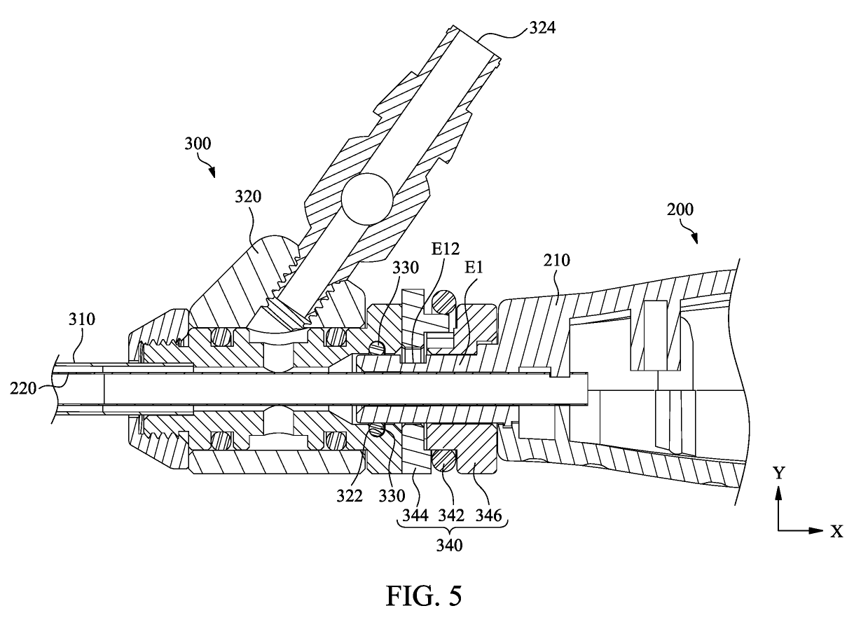

Reference is made to FIGS. 1, 2 and 5 together, where FIG. 5 is a cross-sectional view of the endoscopy device 100 of FIG. 1 taken along line 5-5. The sleeve assembly 300 of the endoscopy device 100 further includes a sleeve attachment portion 320, a sealing ring 330, and an elastic limiting assembly 340. The sleeve attachment portion 320 is connected to the sleeve 310 and has an inner sidewall 322 and a water inlet 324. The sealing ring 330 is embedded in the inner sidewall 322. When the sleeve assembly 300 is disposed at the first end E1 of the endoscope 200, the sealing ring 330 seals a gap between the sleeve attachment portion 320 and the first end E1 of the endoscope 200. Furthermore, the first end E1 of the endoscope 200 includes a recess E12. The elastic limiting assembly 340 is connected to the sleeve attachment portion 320 and includes an elastic member 342, a pressing plate 344, and a connection portion 346. The elastic member 342 is annular. The pressing plate 344 is connected to the elastic member 342 and is movable between a first position and a second position. The connection portion 346 is connected to the pressing plate 344 and the first end E1. The pressing plate 344 is limited by the elastic member 342, so that the pressing plate 344 at the first position engages with the recess E12. In one embodiment, the sealing ring 330 may be an O-ring structure. Thus, the present invention utilizes the sealing ring 330 to seal an opening of the sleeve assembly 300 to prevent water leakage from the inside.

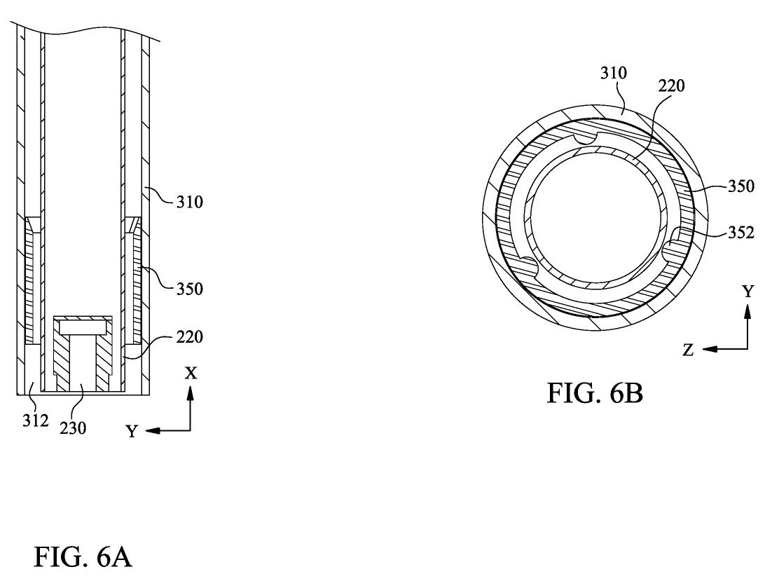

Reference is made to FIGS. 1, 2 and 6A together, where FIG. 6A is a cross-sectional view of the endoscopy device 100 of FIG. 1 taken along line 6A-6A. A camera 230 is disposed inside the front end of the endoscope tube 220 of the endoscope 200, and the camera 230 is configured to capture the image in front of the endoscope tube 220. The sleeve assembly 300 further includes a liner sleeve 350. The liner sleeve 350 is disposed in an internal space of the sleeve 310, and the liner sleeve 350 is configured to limit the endoscope tube 220. The liner sleeve 350 is close to a front end of the sleeve 310. In one embodiment, the endoscope 200 can be a disposable endoscope, and the camera 230 can use a complementary metal oxide semiconductor (CMOS) photosensitive element. However, the present invention is not limited thereto.

Reference is made to FIGS. 1, 5, 6A and 6B together, where FIG. 6B is a cross-sectional view of the endoscopy device 100 of FIG. 1 taken along line 6B-6B. The liner sleeve 350 includes a plurality of protrusions 352. The protrusions 352 protrude toward an interior of the liner sleeve 350 and are configured to abut against an outer wall of the endoscope tube 220 to limit the endoscope tube 220. A water outlet area is formed between the liner sleeve 350 and the endoscope tube 220. Water can enter from the water inlet 324 of the sleeve attachment portion 320, then pass through the water outlet area, and finally flow out from a front water outlet 312 of the sleeve 310. In the present embodiment, the front ends of the endoscope tube 220 and the sleeve 310 are flat ends; and in other embodiments, the front ends of the endoscope tube 220 and the sleeve 310 may be beveled ends, but the present invention is not limited thereto. Thus, the present invention utilizes the protrusions 352 of the liner sleeve 350 to center and position the endoscope tube 220 at the front end of the sleeve 310, thereby increasing the operational stability of the endoscopy device 100.

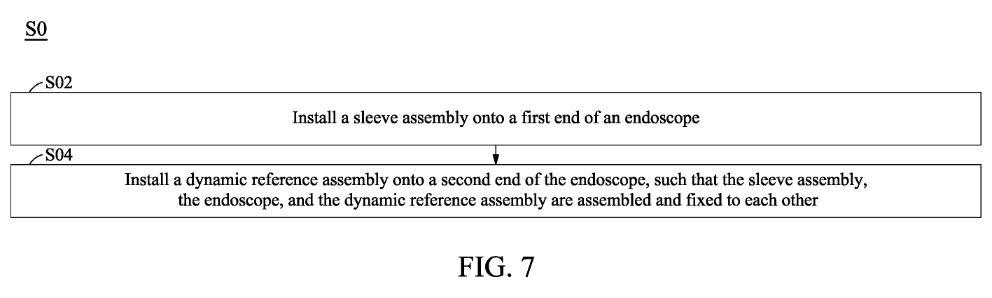

Reference is made to FIGS. 1, 2 and 7 together, where FIG. 7 is a schematic flowchart illustrating an assembly method S0 of an endoscopy device according to a second embodiment of the present invention. The assembly method S0 of the endoscopy device can be applied to the endoscopy device 100 of FIG. 1. The assembly method S0 of the endoscopy device includes performing steps S02, S04. Step S02 includes installing a sleeve assembly 300 onto a first end E1 of an endoscope 200. Step S04 includes installing a dynamic reference assembly 400 onto a second end E2 of the endoscope 200, such that the sleeve assembly 300, the endoscope 200, and the dynamic reference assembly 400 are assembled and fixed to each other. The endoscopy device 100 is configured corresponding to an optical tracker. The first end E1 is opposite to the second end E2. The sleeve assembly 300 is detachably disposed at the first end E1 of the endoscope 200. The sleeve assembly 300 includes a sleeve 310. The sleeve 310 is curved (the sleeve 310 is a curved tube). The second end E2 of the endoscope 200 is away from the sleeve assembly 300, and the sleeve assembly 300 sleeves the first end E1 of the endoscope 200. The dynamic reference assembly 400 is rotatably positioned at the second end E2 of the endoscope 200, and is configured to be detected by the optical tracker to obtain a position of the endoscope 200.

In other embodiments, step S04 may be performed before step S02, and the present invention is not limited thereto. Accordingly, through a bending direction of the curved tube, the assembly method S0 of the endoscopy device of the present disclosure facilitates a physician in finding orientation and direction during use, making it highly suitable for surgical endoscopic operations.

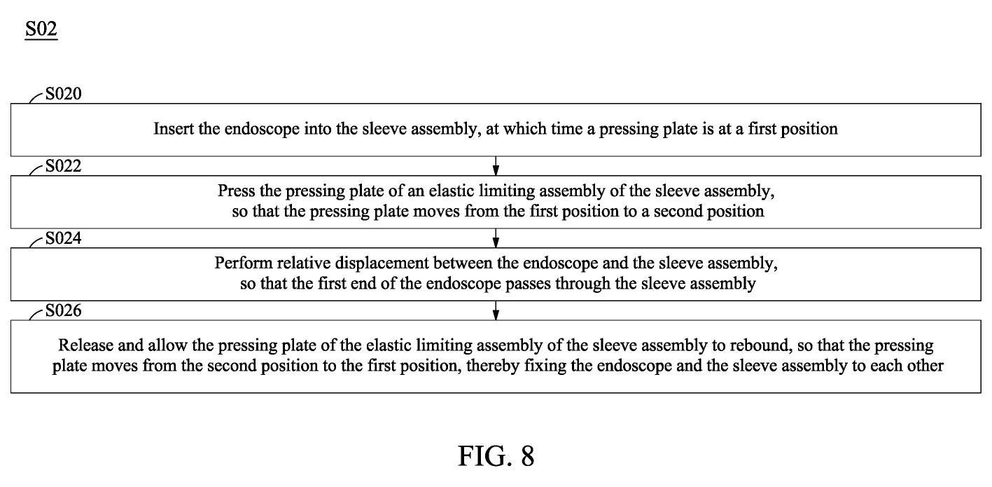

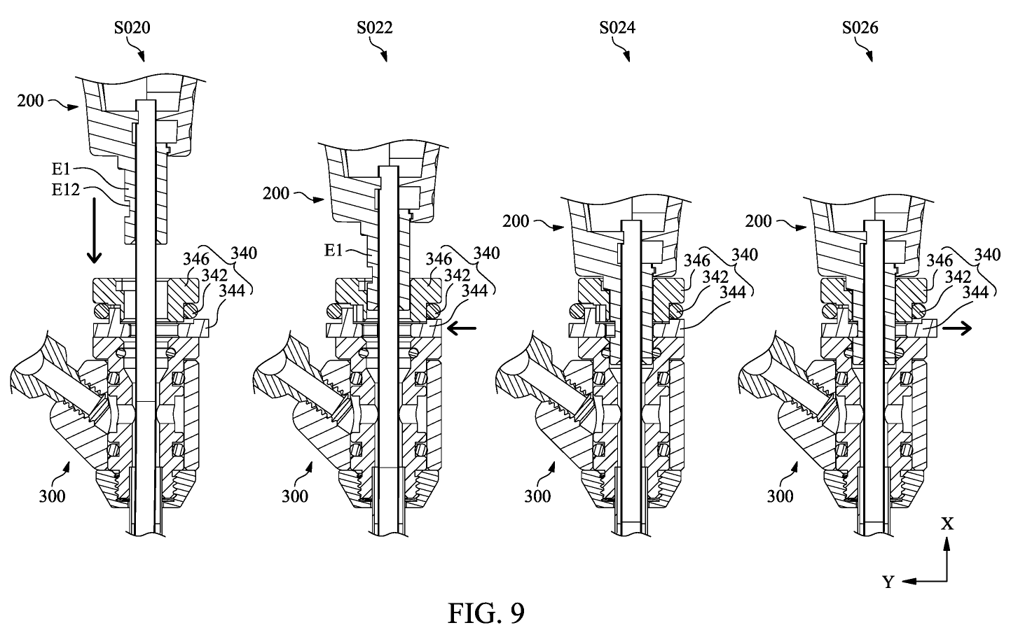

Reference is made to FIGS. 1, 2, 5, 7, 8 and 9 together, where FIG. 8 is a flowchart illustrating one step S02 of the assembly method S0 of the endoscopy device of FIG. 7; and FIG. 9 is a schematic assembly diagram illustrating step S02 of FIG. 8. Step S02 may include performing steps S020, S022, S024, S026. Step S020 includes inserting the endoscope 200 into the sleeve assembly 300, at which time the pressing plate 344 is at a first position, and the first end E1 of the endoscope 200 is blocked by the pressing plate 344 and cannot be fully inserted into the sleeve assembly 300. Step S022 includes pressing the pressing plate 344 of an elastic limiting assembly 340 of the sleeve assembly 300 (e.g., a user presses the pressing plate 344 by hand), so that the pressing plate 344 moves from the first position to a second position. At this time, the pressing plate 344 is at the second position, and the first end E1 of the endoscope 200 can be fully inserted into the sleeve assembly 300. Step S024 includes performing relative displacement between the endoscope 200 and the sleeve assembly 300 (inserting the endoscope 200 to engage with the sleeve assembly 300), so that the first end E1 of the endoscope 200 passes through the sleeve assembly 300. At this time, the pressing plate 344 remains pressed and is at the second position. Step S026 includes releasing and allowing the pressing plate 344 of the elastic limiting assembly 340 of the sleeve assembly 300 to rebound, so that the pressing plate 344 moves from the second position to the first position, thereby fixing the endoscope 200 and the sleeve assembly 300 to each other. The first end E1 of the endoscope 200 includes a recess E12. When the pressing plate 344 moves from the second position to the first position (released and rebounded), the pressing plate 344 at the first position is engaged with the recess E12.

In other embodiments, the endoscope tube and the sleeve of the endoscopy device may be curved straight tubes, that is, the endoscope tube and the sleeve are straight. The assembly method S0 of the endoscopy device and its step S02 in FIGS. 8 and 9 can be applied to a straight-tube type endoscopy device, and the present invention is not limited thereto.

As can be seen from the above implementations, the present invention has the following advantages: First, through the bending direction of the curved tube, a physician can quickly and correctly find orientation and direction during use, which is highly suitable for surgical endoscopic operations, thus solving the issues that the straight-tube structures in the prior art are difficult to identify the correct orientation and direction during an actual operation, thereby reducing operational convenience. The curved tube structure can preserve core advantages of angled lens while also preserving advantage of the small diameter of 0-degree lens. Second, by employing the rotatable dynamic reference assembly, users can adaptively adjust the direction of the dynamic reference frame according to their own usage habits, thereby significantly enhancing operational convenience. Third, by using the protrusions of the liner sleeve, the endoscope tube can be centered and positioned at the front end of the sleeve, thereby increasing the operational stability of the endoscopy device. Fourth, by using the sealing ring to seal the opening of the sleeve assembly, water leakage from the inside is prevented.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. An endoscopy device, configured corresponding to an optical tracker, the endoscopy device comprising:
an endoscope, having a first end and a second end, the first end being opposite to the second end;
a sleeve assembly, detachably disposed at the first end of the endoscope, the sleeve assembly comprising a sleeve, the sleeve being curved; and
a dynamic reference assembly, rotatably positioned at the second end of the endoscope and configured to be detected by the optical tracker to obtain a position of the endoscope;
wherein when the sleeve assembly is disposed at the first end of the endoscope, the second end of the endoscope is away from the sleeve assembly, and the sleeve assembly sleeves the first end of the endoscope.

2. The endoscopy device of claim 1, wherein the endoscope comprises:
a body, having the first end and the second end; and
an endoscope tube, connected to the first end of the body and being curved, and having a shape being identical to that of the sleeve.

3. The endoscopy device of claim 2, wherein the sleeve assembly further comprises:
a liner sleeve, disposed in an internal space of the sleeve, the liner sleeve being configured to limit the endoscope tube.

4. The endoscopy device of claim 3, wherein the liner sleeve comprises:
a plurality of protrusions, protruding toward an interior of the liner sleeve and configured to abut against an outer wall of the endoscope tube to limit the endoscope tube;
wherein a water outlet area is formed between the liner sleeve and the endoscope tube.

5. The endoscopy device of claim 1, wherein the dynamic reference assembly comprises:
a rotating ring; and
a dynamic reference frame, connected to the rotating ring and having a reference surface, the endoscope having a normal direction, an included angle being formed between the reference surface and the normal direction, and the included angle being greater than 0 degrees and less than 90 degrees.

6. The endoscopy device of claim 5, wherein,
the rotating ring comprises at least one positioning bead; and
the second end of the endoscope comprises a groove, and when the dynamic reference assembly is positioned at the second end of the endoscope, the at least one positioning bead is correspondingly embedded in the groove.

7. The endoscopy device of claim 6, wherein a number of the at least one positioning bead is plural, and the rotating ring further comprises:
an annular body, the plurality of positioning beads being spaced apart in the annular body, and the dynamic reference frame being fixedly connected to the annular body.

8. The endoscopy device of claim 1, wherein the sleeve assembly further comprises:
a sleeve attachment portion, connected to the sleeve and having an inner sidewall; and
a sealing ring, embedded in the inner sidewall;
wherein when the sleeve assembly is disposed at the first end of the endoscope, the sealing ring seals a gap between the sleeve attachment portion and the first end of the endoscope.

9. The endoscopy device of claim 8, wherein the first end of the endoscope comprises a recess, and the sleeve assembly further comprises:
an elastic limiting assembly, connected to the sleeve attachment portion and comprising:
an elastic member; and
a pressing plate, connected to the elastic member and movable between a first position and a second position;
wherein the pressing plate is limited by the elastic member, so that the pressing plate at the first position is engaged with the recess.

10. The endoscopy device of claim 1, wherein when the dynamic reference assembly is positioned at the second end of the endoscope, the dynamic reference assembly is detected by the optical tracker to obtain the position of the endoscope.

11. An assembly method of an endoscopy device, comprising:
installing a sleeve assembly onto a first end of an endoscope; and
installing a dynamic reference assembly onto a second end of the endoscope, such that the sleeve assembly, the endoscope, and the dynamic reference assembly are assembled and fixed to each other;
wherein the endoscopy device is configured corresponding to an optical tracker, the first end is opposite to the second end, the sleeve assembly is detachably disposed at the first end of the endoscope, the sleeve assembly includes a sleeve, the sleeve is curved, the second end of the endoscope is away from the sleeve assembly, the sleeve assembly sleeves the first end of the endoscope, and the dynamic reference assembly is rotatably positioned at the second end of the endoscope, and is configured to be detected by the optical tracker to obtain a position of the endoscope.

12. The assembly method of claim 11, wherein step of installing the sleeve assembly onto the first end of the endoscope comprises:
pressing a pressing plate of an elastic limiting assembly of the sleeve assembly, so that the pressing plate moves from a first position to a second position;
performing relative displacement between the endoscope and the sleeve assembly, so that the first end of the endoscope passes through the sleeve assembly; and
releasing and allowing the pressing plate of the elastic limiting assembly of the sleeve assembly to rebound, so that the pressing plate moves from the second position to the first position, thereby fixing the endoscope and the sleeve assembly to each other;
wherein the first end of the endoscope comprises a recess, and when the pressing plate moves from the second position to the first position, the pressing plate at the first position is engaged with the recess.

13. The assembly method of claim 11, wherein the endoscope comprises:
a body, having the first end and the second end; and
an endoscope tube, connected to the first end of the body and being curved, and having a shape being identical to that of the sleeve.

14. The assembly method of claim 13, wherein the sleeve assembly further comprises:
a liner sleeve, disposed in an internal space of the sleeve, the liner sleeve being configured to limit the endoscope tube.

15. The assembly method of claim 14, wherein the liner sleeve comprises:
a plurality of protrusions, protruding toward an interior of the liner sleeve and configured to abut against an outer wall of the endoscope tube to limit the endoscope tube;
wherein a water outlet area is formed between the liner sleeve and the endoscope tube.

16. The assembly method of claim 11, wherein the dynamic reference assembly comprises:
a rotating ring; and
a dynamic reference frame, connected to the rotating ring and having a reference surface, the endoscope having a normal direction, an included angle being formed between the reference surface and the normal direction, and the included angle being greater than 0 degrees and less than 90 degrees.

17. The assembly method of claim 16, wherein:
the rotating ring comprises at least one positioning bead; and
the second end of the endoscope comprises a groove, and when the dynamic reference assembly is positioned at the second end of the endoscope, the at least one positioning bead is correspondingly embedded in the groove.

18. The assembly method of claim 17, wherein a number of the at least one positioning bead is plural, and the rotating ring further comprises:
an annular body, the plurality of positioning beads being spaced apart in the annular body, and the dynamic reference frame being fixedly connected to the annular body.

19. The assembly method of claim 11, wherein the sleeve assembly further comprises:
a sleeve attachment portion, connected to the sleeve and having an inner sidewall; and
a sealing ring, embedded in the inner sidewall;
wherein when the sleeve assembly is disposed at the first end of the endoscope, the sealing ring seals a gap between the sleeve attachment portion and the first end of the endoscope.

20. The assembly method of claim 11, wherein step of installing the dynamic reference assembly onto the second end of the endoscope comprises:
when the dynamic reference assembly is positioned at the second end of the endoscope, the dynamic reference assembly is detected by the optical tracker to obtain the position of the endoscope.
